(19) United States
(12) Patent Application Publication
(10) Pub. No.: US 2026/0264363 A1
(43) Pub. Date: Sep. 10, 2026

(54) NONWOVEN AGGREGATE AND METHOD OF MAKING SAME

(71) Applicants: OPTIMUM TECHNOLOGY IP LLC, Douglas (GB); REIFENHÄUSER GMBH & CO. KG MASCHINENFABRIK, Troisdorf (DE)

(72) Inventors: Kim PATCHETT, Grays, Essex (GB); Patrick BOHL, Hennef (DE); Michael MASS, Roesrath (DE); Sebastian SOMMER, Troisdorf (DE); Tobias WAGNER, Koeln (DE)

(21) Appl. No.: 18/727,585

(22) PCT Filed: Jan. 20, 2023
(86) PCT No.: PCT/EP2023/051446

§ 371 Date: Jul. 9, 2024

(30) Foreign Application Priority Data
Jan. 28, 2022 (DE) 10 2022 102 085.1

(51) Int. Cl. B32B 5/02; B32B 5/08; B32B 5/26; B32B 37/14

(52) U.S. Cl. CPC B32B 5/022; B32B 5/08; B32B 5/267; B32B 37/14; B32B 2250/03; B32B 2250/20; B32B 2260/023; B32B 2262/124

(57) ABSTRACT

The invention relates to a nonwoven unit comprising at least one nonwoven layer having at least one first layer and at least one second layer. The second layer is in the form of a nonwoven layer composed of continuous filaments. The second layer has absorption particles which are embedded in the pores between the filaments of the second layer. At least one third layer, which is applied to the second layer and is in the form of a nonwoven layer composed of continuous filaments, or at least one second nonwoven layer, which is applied to the second layer and is composed of continuous filaments, is provided. The third layer or the second nonwoven layer has hydrophilic properties. The first layer forms a barrier preventing the penetration of particles or absorption particles having a size greater than 150 μm.

The invention relates to a nonwoven aggregate having at least one nonwoven lamina with at least one first layer and at least one second layer, where the second layer is a nonwoven layer of continuous filaments. The nonwoven aggregate has embedded absorption particles, in particular for absorbing fluid media. In addition, the invention relates to a method and an apparatus for making a nonwoven aggregate.

Nonwoven aggregates of the above-mentioned type are generally known in practice in various embodiments. Such nonwoven aggregates are used in hygiene products, for example in diapers, incontinence products and feminine hygiene products. The absorbent particles have absorbent properties, in particular toward fluid media, preferably toward aqueous fluid media, for example urine and other body fluids. In such nonwoven aggregates with embedded absorption particles, it is desirable that the properties are set in such a way that the nonwoven aggregate does not allow a fluid medium from the interior of the nonwoven aggregate to escape from a first nonwoven aggregate outer face or lower face and that the nonwoven aggregate passes a fluid medium to the embedded absorption particles sufficiently quickly through the other nonwoven aggregate outer face or upper face opposite the first nonwoven aggregate outer face. If such nonwoven aggregates are thus exposed to fluid media on the upper face, these fluid media should penetrate as quickly as possible to the embedded absorption particles and from there no longer penetrate further through the nonwoven aggregate or emerge from it.

In addition, the absorption particles put into the nonwoven aggregate should move as little as possible within the nonwoven aggregate and thus be securely fixed in the nonwoven aggregate. Absorption particles should also be prevented from falling out of the nonwoven aggregate as far as possible.

With the nonwoven aggregates known from practice, these properties could not be realized in a functionally reliable and simple way so far. In particular, it is not possible to adjust the properties of the nonwoven aggregate with regard to the passage of fluid media and at the same time to ensure the fixation of the absorption particles within the nonwoven aggregate with the known measures, or not reliably and with reasonable effort. Although the fixation of absorption particles in nonwoven aggregates by binders or adhesives is known, this is complex and can influence the properties of the embedded absorption particles in an undesirable way. In addition, the binders or adhesives can cause skin irritation or allergies in the event of skin contact. Overall, there is a need for improvement in this respect. This is where the invention comes in.

In contrast, the object of the invention is to provide a nonwoven aggregate of the above-mentioned type whose properties with regard to the passage of fluid media can be adjusted simply and reliably in such a way that the nonwoven aggregate fulfills all requirements and that, in particular, fluid media can pass from an outer or upper face of the nonwoven aggregate to the embedded absorption particles and from there cannot pass further through the nonwoven aggregate or escape from the nonwoven aggregate and, preferably, furthermore, that a secure In particular, fluid media can reach the embedded absorption particles from the outside or upper face of the nonwoven aggregate and from there do not pass through the nonwoven aggregate or exit the nonwoven aggregate, and it is preferably still possible to securely fix the absorption particles within the nonwoven aggregate. Furthermore, the invention has the further object of creating a method of making such a nonwoven aggregate and an apparatus for making such a nonwoven aggregate.

To attain these objects, the invention teaches a nonwoven aggregate with at least one nonwoven lamina with at least one first layer and at least one second layer, the second layer being in the form of a nonwoven layer of continuous filaments, wherein
the second layer comprises absorption particles, in particular for absorbing fluid media, embedded in the pores between the filaments of the second layer,
at least one third layer in the form of a nonwoven layer of continuous filaments is applied to the second layer, in particular directly applied, or at least one second nonwoven lamina of continuous filaments is applied to the second layer, in particular directly applied,
the third layer or the second nonwoven lamina has hydrophilic properties, in particular more hydrophilic properties than the first layer, and
the first layer forms a barrier against penetration by particles or absorption particles with a size greater than 150 μm, preferably greater than 100 μm, preferably greater than 50 μm, very preferably greater than 25 μm, particularly preferably greater than 10 μm and very particularly preferably greater than 0.5 μm.

When the term “layers” of filaments or continuous filaments or a nonwoven lamina with layers of filaments or continuous filaments is used here or in the following, this means that the layers of filaments or continuous filaments are spun from only one and the same spinning beam. When the term “laminae” or nonwoven laminae of filaments or continuous filaments is used here or below, this means that the individual laminae are each produced with a separate spinning beam. Endless filaments differ from staple fibers, which have significantly shorter lengths of 10 mm to 60 mm for example as opposed to their virtually endless length.

According to the invention, the third layer or the second nonwoven lamina has hydrophilic properties, in particular more hydrophilic properties than the first layer. In the context of the invention, the fact that a layer or a lamina is more hydrophilic in terms of its properties or is more hydrophilic than another layer or lamina preferably means that the layer or lamina has more hydrophilic properties with respect to an aqueous sodium-chloride solution, preferably a 0.9% aqueous sodium-chloride solution, than the other layer or lamina. A 0.9% aqueous sodium-chloride solution in the context of the invention means in particular a 0.9 wt % solution of sodium chloride in deionized or distilled water. The measurement or determination of the hydrophilic properties of a layer or lamina of the nonwoven aggregate can be determined by determining the surface tension of the surface or the outside of the layer or lamina with respect to a test liquid, in particular with respect to a 0.9% aqueous sodium-chloride solution, by determining the time until this test liquid has penetrated into the layer or lamina. For this purpose, the nonwoven aggregate may first be separated into its individual layers and/or laminae. The layer or lamina into which the test liquid penetrates faster has the more hydrophilic properties or is more hydrophilic. In addition or alternatively, the contact angle between a test liquid and the layer can be determined in order to determine whether a layer has more hydrophilic properties or is more hydrophilic than another layer. Preferably, a 0.9% aqueous sodium-chloride solution can also be used as the test liquid. If, when using a test liquid, no time difference in the penetration of the test liquid can be determined when comparing two layers or laminae, the wetting tension of the layers or laminae is preferably determined in accordance with DIN ISO 8296 “Plastics films and sheets—Determination of wetting tension.”

According to the invention, the first layer forms a barrier against the penetration of particles or absorption particles with a size greater than 150 μm, preferably greater than 100 μm, preferably greater than 50 μm, very preferably greater than 25 μm, particularly preferably greater than 10 μm and very particularly preferably greater than 0.5 μm. This is achieved in particular by the average pore size of pores formed in the first layer and/or by the consolidation or final consolidation of the nonwoven aggregate. The average pore size is an equivalent for the density of the nonwoven structure and/or the barrier resistance to the penetration of particles of the specified size or size distribution. The fact that a layer or a lamina forms a barrier against the penetration of particles or absorption particles means in particular that this layer or lamina prevents or substantially prevents the passage of particles of the specified size. Moreover, it is within the scope of the invention that the nonwoven aggregate according to the invention is a consolidated or final-consolidated nonwoven aggregate or a nonwoven laminate.

It is within the scope of the invention that the mean pore size of pores formed in the first layer is smaller than the mean pore size of the pores between the filaments of the second layer and/or that the third layer or the second nonwoven lamina has a mean pore size of the pores between the filaments that is smaller than the mean pore size of the pores between the filaments of the second layer. Expediently, the mean pore size of the first layer corresponds to a maximum of 50%, preferably a maximum of 25%, preferably a maximum of 15% of the mean pore size of the second layer.

In the context of the invention, the term average pore size refers in particular to the average pore size or the average pore volume of the pores. In the context of the invention, the average pore size or the ratio of the average pore size of two layers, of two layers or of one layer and one layer according to one embodiment can be determined by porometry using a porosimeter or an autoporosimeter. For this purpose, the nonwoven aggregate is preferably separated into its individual layers and/or laminae and the pore-size distribution of the pores of the individual layers and/or laminae is determined by porometry. Reference is made to WO 2011/053677 [U.S. Pat. No. 9,714,484] and in particular to pages 18 to 20. Additionally or alternatively, the mean pore size or the ratio of the mean pore size of two layers, two laminae or one layer and one lamina can preferably be determined by microcomputed tomography (CT) by three-dimensional scanning. As with conventional X-ray computed tomography, microcomputed tomography allows nondestructive testing of materials and components with regard to local differences in density. This allows pores, blowholes and cracks as well as different phases and components to be detected and analyzed. For this purpose, a virtual 3D model of the material or component is recreated from radiographs at different angles using 3D reconstruction. In contrast to medical tomography, the resolution can be in the micrometer range depending on the sample or component size. Microcomputed tomography is particularly suitable for examining fibrous materials, their structure and gradients. For this purpose, a preferably cubic sample with an edge length of 8 mm for example is taken from the nonwoven aggregate and scanned with a resolution of 4 μm voxel size, for example. A digital twin of the material sample can be calculated from the raw scan data obtained. Reference is made to WO 2020/103964 [US 2022/0008263] and in particular to pages 49 to 53. The structural model or digital twin can preferably be generated using the FiberFind module from Math2Market GmbH. The pores or cavities between the fibers can be measured on a structural model or digital twin generated in this way. Preferably, modules such as MathDict and/or PoroDict from Math2Market GmbH, which can be used to analyze the properties of porous media, can be used for this purpose. In this way, the average pore size or the ratio of the average pore sizes of the layers or laminae in the nonwoven aggregate according to the invention can be determined. In addition or as an alternative to determining the pore size, a gray-value analysis of sections through a uCT model can be carried out for the relevant layers within the scope of the invention in order to determine the density of the nonwoven structure in a simplified manner. For this purpose, a certain number of sections are placed across the thickness, for example five or ten sections, the grey values of each section are binarized in black and white (0/255) using a threshold value method and then the mean grey value of the layers is determined. Finally, the average gray values of the different slices can be compared. The higher (brighter) the gray value, the denser the fleece structure.

It is preferred that the third layer or the second nonwoven lamina has an average pore size of the pores between the filaments that is smaller than the average pore size of the pores between the filaments of the second layer. Preferably, the third layer or the second nonwoven lamina forms a barrier against the penetration of particles or absorption particles with a size greater than 150 μm, preferably greater than 100 μm, preferably greater than 50 μm, very preferably greater than 25 μm, particularly preferably greater than 10 μm and very particularly preferably greater than 0.5 μm. In the nonwoven aggregate according to the invention, it is possible that an intermediate layer of continuous filaments or an intermediate layer of continuous filaments is or applied between the second layer and the third layer or the second nonwoven lamina. According to one embodiment, the intermediate layer or intermediate layer can have an average pore size of the pores between the filaments that is larger than the average pore size of the pores between the filaments of the second layer. With regard to the third layer, it is also possible that this third layer is produced by partial consolidation, in particular by thermal partial consolidation, of the outer face of the second layer directed toward the outside of the nonwoven aggregate.

According to a preferred embodiment of the invention, no absorption particles or essentially no absorption particles are embedded in the pores of the third layer or in the pores of the second nonwoven lamina. It is possible that absorption particles are embedded in the first layer or in the pores of the first layer. Such absorption particles embedded in the pores of the first layer are expediently absorption particles whose average size is smaller than the average size of the absorption particles embedded in the pores of the second layer. According to the embodiment in which absorption particles are also embedded in the pores of the first layer, the first layer thus acts both as a barrier and as a layer for embedding absorption particles of small size.

It has been proven effective when the third layer or the second nonwoven lamina is an outwardly closing layer or lamina of the nonwoven aggregate. According to another preferred embodiment, the third layer or the second nonwoven lamina is followed by a further top layer of continuous filaments or a top layer of continuous filaments. It is also preferred that the first layer of the nonwoven aggregate is an outwardly closing layer of the nonwoven aggregate. In this way, the nonwoven aggregate according to the invention is preferably terminated by the first layer toward a first nonwoven aggregate outer face or lower face and terminated by the third layer or the second nonwoven lamina toward a second nonwoven aggregate outer face or upper face, in particular opposite the first nonwoven aggregate outer face. Alternatively, the nonwoven aggregate according to the invention is preferably terminated by the first layer toward a first nonwoven aggregate outer or top face and terminated by a top layer of continuous filaments or a top layer of continuous filaments toward the second nonwoven aggregate outer face or bottom side.

A particularly preferred embodiment of the nonwoven aggregate according to the invention is characterized in that the absorption particles are embedded in the pores of the second layer without adhesives or binders. In the context of the invention, the fact that the absorption particles are embedded in the pores of the second layer without adhesives or binders means in particular that there is no binder or adhesive provided for fixing the absorption particles in the pores of the second layer. In particular, no adhesive or hot-melt adhesive for fixing the absorption particles in the pores of the second layer is present in the nonwoven aggregate. In the context of the invention, a low-melting-point binder of multicomponent filaments, in particular of bicomponent filaments, is not regarded as an adhesion agent or binder or adhesive or hot-melt adhesive. In the context of the invention, the absorption particles are preferably fixed in the pores of the second layer or in the nonwoven aggregate by the configuration of the layers of the aggregate and their properties, as well as the subsequent consolidation or final consolidation of the nonwoven aggregate. The embodiment, according to which the absorption particles are embedded in the pores of the second layer without adhesives or binders, is based on the discovery that undesirable impairment of the functionality of the absorption particles by adhesives or binders is avoided. In addition, skin irritations or allergies, which can sometimes result from skin contact with a binder or adhesion agent, can be avoided. It should also be understood that the entire nonwoven aggregate is preferably free of adhesives or binders and, in this respect, no adhesives or binders are present in the nonwoven aggregate to fix the absorption particles in the pores of the nonwoven aggregate.

It is within the scope of the invention that the absorption particles are absorption particles for absorbing fluid media. The fluid media may expediently be water-based fluid media.

In one embodiment, such a fluid medium may be urine. According to one embodiment of the invention, the absorbent particles are superabsorbent particles. Preferably, the absorbent particles are a hydrogel. Preferably, the absorption particles are based on sodium polyacrylate. Further preferably, the absorption particles consist of sodium polyacrylate or essentially of sodium polyacrylate.

According to a preferred embodiment of the invention, the absorption particles have an average particle size of from 10 μm to 3000 μm, preferably from²⁰ μm to 2500 μm, particularly preferably from 40 μm to 2000 μm, most preferably from 50 μm to 1250 μm and most preferably from 100 μm to 1000 μm.

A particularly preferred embodiment of the nonwoven aggregate according to the invention is characterized in that the third layer or the second nonwoven lamina has more hydrophilic properties than the second layer. This embodiment is based on the discovery that the third layer or the second nonwoven lamina can then pass this fluid medium through to the second layer quickly and reliably when this layer or lamina is exposed to a fluid medium, so that the fluid medium can quickly reach the absorption particles embedded in the second layer. It has been proven effective when the second layer has more hydrophilic properties than the first layer. Preferably, the hydrophilic properties of the nonwoven aggregate increase across the thickness of the nonwoven aggregate, i.e. in the direction transverse, in particular perpendicular or essentially perpendicular to its planar extension from a first nonwoven aggregate outer face or lower face to a second nonwoven aggregate outer face opposite the first nonwoven aggregate outer face or upper face. According to one embodiment, the first nonwoven aggregate outer face or lower face can be formed by the first layer and preferably the second nonwoven aggregate outer face or upper face is then formed by the third layer or the second nonwoven lamina. This embodiment is based on the discovery that the third layer or the second nonwoven lamina is then preferably the layer of the aggregate comprising the third layer or second nonwoven lamina, the second layer and first layer that has the most hydrophilic properties. A fluid medium applied to the third layer or the second nonwoven lamina, can then be quickly passed through to the second nonwoven lamina and reach the absorption particles embedded in the second nonwoven lamina. Further passage of the fluid medium through the nonwoven aggregate is then prevented by the least hydrophilic first layer, so that the first layer acts as a kind of barrier against the fluid medium passing through.

It is possible for the hydrophilic properties of a layer or lamina to be adjusted by admixing a hydrophilic additive to the melt from which the filaments are spun and/or by applying a hydrophilizing agent, in particular a hydrophilic lubricant, to the layer or lamina after spinning. In the context of the invention, the fact that a hydrophilizing agent is or is applied to a lamina or layer means in particular that the hydrophilizing agent is applied to the filaments or the deposited filaments of the lamina or layer. By adding a hydrophilic additive to the melt from which the filaments are spun, the fibers and thus indirectly the lamina or layer are given more hydrophilic properties. Applying a hydrophilic additive or a hydrophilic lubricant to the layer or lamina after spinning gives the layer or lamina more hydrophilic properties, but the hydrophilic properties of the filament material are not changed.

It is particularly preferred that at least one hydrophilizing agent, in particular a hydrophilic lubricant, is applied to the third layer or the second nonwoven lamina and/or to the second layer. In the context of the invention, the hydrophilizing agent or hydrophilic lubricant applied can accelerate the passage of a fluid medium, in particular an aqueous medium, for example urine or the like, through the layer or lamina. In particular, the hydrophilic properties of a layer are adjusted by the hydrophilizing agent or the hydrophilic avivage. As a result, the liquid strike-through time for passage of a fluid medium or for the passage of an aqueous fluid medium can be shortened. This is explained in more detail below.

The hydrophilizing agent or the hydrophilic lubricant is preferably a liquid hydrophilizing agent that conveniently dries or is dried after application. It is preferred that the hydrophilizing agent, in particular the hydrophilic lubricant, contains at least one carboxyl compound or at least one organic carboxyl compound.

Preferably, the hydrophilizing agent or the hydrophilic lubricant contains at least one ester of a carboxylic acid or a fatty acid ester. In the context of the invention, at least one component selected from the group “S+S Silastol PHP 207, S+S Silastol PHP 163, Pulcra Chemicals Stantex S 6327” can be used as hydrophilizing agent or hydrophilic lubricant.

It is possible that the first layer of the nonwoven aggregate according to the invention is a film, in particular a plastic film. According to a further preferred embodiment, the first layer can be a separate base nonwoven layer produced with a separate spinning beam. The base nonwoven layer can be formed from continuous filaments. It is possible that the base nonwoven layer is a spunbond laminate and/or as a meltblown lamina. In principle, the first layer can also be an laminate of at least one film or plastic film and a base nonwoven layer of filaments or continuous filaments, whereby the base nonwoven layer can be a meltblown layer and/or a spunbond layer.

According to a particularly preferred embodiment of the nonwoven aggregate according to the invention, the first layer is a nonwoven layer of continuous filaments. Preferably, the first layer and/or the second layer and/or the third layer is a spunbond nonwoven layer and/or the second nonwoven lamina is a spunbond nonwoven layer. In the context of the invention, however, a nonwoven layer of continuous filaments or a nonwoven layer of continuous filaments can also be a meltblown nonwoven layer or as a meltblown nonwoven layer. Preferably, the first layer and/or the second layer and/or the third layer is a spunbond nonwoven layer and/or the second nonwoven lamina is a spunbond nonwoven lamina. It has been proven effective when the first layer and the second layer and the third layer or the second nonwoven lamina are a spunbond nonwoven layer or as a spunbond nonwoven lamina.

It is particularly preferred that the first nonwoven layer or the first layer and the second layer are spun from only one spinning beam or from only one spinneret. In this case, the first layer and the second layer are a nonwoven layer made of continuous filaments and the first nonwoven layer is a nonwoven layer made of continuous filaments. If two layers are spun from only one spinning beam or from only one spinneret, it is preferred that the filaments are spun from this one spinning beam or from this one spinneret in such a way that the properties of the deposited filaments differ in such a way that two nonwoven layers of continuous filaments with different properties result or are deposited on top of each other. This can be achieved by varying the properties of the spun filaments in the machine direction (MD direction) and/or transverse to the machine direction (CD direction), preferably only in the machine direction (MD direction), along the spinning beam or the spinneret. This is explained in more detail below. In this context, it is preferable that the properties of the filaments spun from only one spinning beam or from only one spinneret are the same or essentially the same in the direction transverse to the machine direction (CD direction), so that the individual deposited layers are homogeneous or essentially homogeneous throughout their entire areas with regard to these filament properties. The fact that two nonwoven laminae of continuous filaments are spun from only one spinning beam or only one spinneret can be recognized in the resulting nonwoven aggregate in particular by a short transition area across the thickness of the nonwoven aggregate, so that there is preferably no sharp transition between the two nonwoven layers, as would be the case with two nonwoven layers of continuous filaments spun from two separate spinning beams. This can also be shown for example by delamination tests of the layers or laminae of a nonwoven aggregate and/or by microcomputed tomography (see above).

A preferred embodiment of the invention is characterized in that the third layer is spun from the same spinning beam or from the same spinneret as the first and second layers. In this case, the third layer is expediently a nonwoven layer of continuous filaments. It is possible that the third layer is produced by partial consolidation, in particular by thermal partial consolidation, of the outer face of the second layer directed toward the outside of the nonwoven aggregate. According to an alternative embodiment, the second nonwoven lamina is spun from a further or second spinning beam or from a further or second spinneret. It is also possible that the second nonwoven lamina is applied to the second layer by an unwinder as a separate nonwoven lamina.

According to the invention, the second layer is a nonwoven layer of continuous filaments. Preferably, the first layer is also a nonwoven layer of continuous filaments. Preferably, the nonwoven aggregate has a third layer that is a nonwoven layer of continuous filaments or the nonwoven aggregate has a second nonwoven lamina of continuous filaments. In this context, it has proven useful for the filaments of the first layer and/or the filaments of the third layer or the second nonwoven lamina to have a lower average titer than the filaments of the second layer. Further preferably, the filaments of the third layer or the second nonwoven lamina have a higher average titer than the filaments of the first layer. According to a particularly preferred embodiment of the nonwoven aggregate according to the invention, the average titer of the filaments of the first layer is 0.1 den to 5 den, preferably 0.2 den to 2.5 den, preferably 0.3 den to 2.0 den, particularly preferably 0.4 den to 1.5 den, most preferably 0.5 den to 1.3 den, for example 1.1 den or about 1.1 den. It has been proven effective when the average titer of the filaments of the second layer is 2.0 den to 12 den, preferably 3.0 den to 10 den, preferably 4.0 den to 8.0 den and particularly preferably 5.5 den to 6.5 den, for example 6.0 den or about 6.0 den. With regard to the third layer or the second nonwoven layer, it is preferred that the average titer of the filaments used is 0.1 den to 5.0 den, preferably 0.2 den to 2.5 den, preferably 0.3 den to 2.0 den, particularly preferably 0.4 den to 1.5 den, most preferably 0.5 den to 1.3 den, for example 1.1 den or about 1.1 den. According to one embodiment, the specified den ranges result in particular when using polypropylene/polyethylene as filament components of bicomponent filaments. Filament titers in μm are then preferably obtained by converting the specified den values assuming a density of 0.92g/cm³.

The embodiment of the nonwoven aggregate according to the invention in which the filaments of the first layer and/or the filaments of the third layer or the second nonwoven lamina have a lower average titer than the filaments of the second layer and preferably the filaments of the third layer or the second nonwoven lamina have a higher average titer than the filaments of the first layer, is based on the discovery that the different titers of the individual layers or laminae allow the different pore sizes of the individual layers or laminae to be adjusted in a functionally reliable manner. In addition, it is preferably ensured that the third layer or the second nonwoven lamina initially enables the injecting absorption particles through this layer into the nonwoven aggregate or into the second layer and then, after the consolidation or final consolidation of the nonwoven aggregate, acts in particular as a barrier, as already described above.

According to a highly recommended embodiment of the invention, the filaments of the first layer and/or the filaments of the second layer and/or the filaments of the third layer or the second nonwoven lamina are multicomponent filaments, in particular bicomponent filaments. Preferably, the multicomponent filaments have at least one low-melting-point binder and particularly preferably the multicomponent filaments are crimped continuous filaments. It has proven to be the case that the filaments of the first layer and the filaments of the second layer and the filaments of the third layer or the second nonwoven lamina are multicomponent filaments, in particular bicomponent filaments, and that the multicomponent filaments preferably have at least one low-melting-point binder and are particularly preferably crimped continuous filaments.

In this context, it has also proven to be the case that the degree of crimp of the filaments of the first layer and/or the filaments of the third layer or the second layer is lower than the degree of crimp of the filaments of the second layer. In the context of the invention, the fact that the degree of crimp of the filaments of one layer or lamina is lower than the degree of crimp of the filaments of another layer or lamina means in particular that the filaments have fewer crimps or bends per centimeter of filament length (number of bends) and/or a larger crimp diameter or bend diameter than the filaments of the other layer or lamina. The number of crimp loops or crimp arcs per centimeter of filament length is measured in particular according to Japanese standard JIS L10151518 by counting the crimps under a bias of 2 mg/den in ( 1/10 mm), based on the stretched length of the filaments. A sensitivity of 0.05 mm is used to determine the number of crimps. The measurement is conveniently carried out with a “Favimat” apparatus from TexTechno, Germany. Please refer to the publication “Automatic Crimp Measurement on Staple Fibers”, Denkendorf Kolloquium, “Textile Mess und Prüftechnik”, 9.11.99, Dr. Ulrich Mörschel (in particular page 4, FIG. 4). The filaments (or the filament sample) are removed as a filament wad before it adheres to the deposit or from the deposition mesh deposition belt before further consolidation and the filaments are separated and measured. The sheet diameter is conveniently measured by placing the nonwoven fabric to be measured under a microscope and generating a still image using suitable magnification, in which the sheet diameter can be measured. In the case of nonwoven aggregates or nonwoven laminates with several layers, the optical system must be focused on the surface of each visible layer so that the other surfaces or layers lie as far as possible outside the depth of field. Due to the random distribution of the filaments or the random distribution of the sheet diameters, at least 25 measurements are required in each case. The arithmetic mean value is specified. This embodiment is based on the discovery that different pore sizes of the layers or laminae can preferably be set due to the different degree of crimp of the layers or laminae. According to one embodiment, the first layer or the first nonwoven layer of continuous filaments has a number of sheets in the range 0 to 3 per cm and the second layer has a number of sheets in the range 2 to 6 per cm, with the second layer preferably having a greater number of sheets than the first layer. For a more open structure, larger crimp sheet amplitudes are particularly preferred.

It has already been explained above that the filaments of the first layer and/or the filaments of the second layer and/or the filaments of the third layer or the second nonwoven lamina are multicomponent filaments, in particular bicomponent filaments, and that the multicomponent filaments expediently have at least one low-melting-point binder. It is within the scope of the invention that the proportion of the low-melting-point binder in the multicomponent filaments of the first layer and/or the third layer or second nonwoven lamina is higher than the proportion of the low-melting-point binder in the multicomponent filaments of the second layer. Expediently, the proportion of the low-melting-point binder component in the multicomponent filaments of the first layer is 10 to 70 wt %, preferably 15 to 65 wt %, preferably 20 to 60 wt %, particularly preferably 35 to 55 wt %, for example 50 wt % or about 50 wt % based on the components of the multicomponent filaments of the first layer. Further preferably, the proportion of the low-melting-point binder component in the multicomponent filaments of the second layer is 5 to 55 wt. %, preferably 10 to 30 wt. %, particularly preferably 15 to 30 wt. %, especially preferably 15 to 25 wt. %, for example 20 wt. % or about 20 wt. % based on the components of the multicomponent filaments of the second layer. With regard to the third layer or the second nonwoven lamina, it is preferred that the proportion of the low-melting-point binder in the multicomponent filaments is 15 wt. % to 60 wt. %, preferably 20 wt. % to 50 wt. %, preferably 22 wt. % to 40 wt. %, for example 30 wt. % or about 30 wt. %, based on the components of the multicomponent filaments of the third layer or the second nonwoven lamina.

The following information regarding the components of the multicomponent filaments preferably applies to all layers or laminae of the nonwoven aggregate of multicomponent filaments, in particular of bicomponent filaments: It is particularly preferred that the at least one low-melting-point binder of the multicomponent filaments consists or essentially consists of polyethylene. If “essentially consists” is stated here or below in relation to the components of the multicomponent filaments or the bicomponent filaments and in particular to a plastic or to a polymer, this means in particular that the component or the polymer is present to at least 95 wt %, preferably to at least 97 wt % and preferably to at least 98 wt %. The remaining percent by weight can be formed in particular by additives such as plasticizers, fillers, dyes and the like. It is further preferred that the at least one further component or the second component of the multicomponent filaments, in particular the bicomponent filaments, consists or essentially consists of polypropylene or of a polyester. By the second or further component is meant in particular the second component present in addition to the low-melting-point binder or a further component present in addition to the low-melting-point binder. The second or further component of the multicomponent filaments or bicomponent filaments then forms the high-melting-point component in particular. For the further or second component, at least one polypropylene copolymer can also be used instead of polypropylene or in addition to polypropylene, and at least one polyester copolymer can also be used instead of the polyester or in addition to the polyester. Polyethylene terephthalate (PET) and/or polylactide (PLA) is particularly suitable as a polyester and PET copolymer (CoPET) is particularly suitable as a polyester copolymer. A recommended

An embodiment of the invention is characterized in that the at least one further component or the second component of the multicomponent filaments or bicomponent filaments consists or essentially consists of at least one plastic from the group “polypropylene, polypropylene copolymer, polyethylene terephthalate (PET), polylactide (PLA), PET copolymer (CoPET) .” According to a particularly preferred embodiment of the invention, the low-melting-point binder of the multicomponent filaments or bicomponent filaments consists of polyethylene or essentially of polyethylene and the second or further component of the multicomponent filaments or bicomponent filaments, in particular the high-melting-point component, consists of polypropylene or essentially of polypropylene.

If, according to a preferred embodiment, the filaments of the second layer are multicomponent filaments, in particular bicomponent filaments, the multicomponent filaments preferably having at least one low-melting-point binder, it has proved particularly useful for the binder proportion of the multicomponent filaments of the second layer to be dimensioned such that in the event of swelling of the absorption particles, in particular as a result of absorption of fluid media, at least partial breaking of the bonds between the filaments of the second layer is possible and where for this purpose the binder proportion of the multicomponent filaments is 5 to 55 wt. wt %, preferably 10 to 30 wt % and in particular 15 to 25 wt % based on the components of the multicomponent filaments. This embodiment is based on the discovery that a sufficiently low proportion of the low-melting-point binder in the multicomponent filaments or bicomponent filaments of the second layer and, in particular, the resulting relatively low consolidating strength when the absorption particles swell, makes it possible to at least partially break open the filament-filament bonds so that the absorption particles do not emerge from the second layer or essentially do not emerge from the second layer during swelling.

It is within the scope of the invention in principle possible for the multicomponent filaments or bicomponent filaments of the layers or laminae of the nonwoven aggregate to be multicomponent filaments or bicomponent filaments with core/sheath configuration, in particular with eccentric core/sheath configuration, with side-by-side configuration, with multilobal configuration, with trilobal configuration or similar cross-sectional configurations.

It is within the scope of the invention that the multicomponent filaments of the first layer and/or of the third layer or of the second nonwoven lamina are multicomponent filaments with a core/sheath configuration, in particular with an eccentric core/sheath configuration, and where according to a preferred embodiment the multicomponent filaments of the second layer are multicomponent filaments with a side-by-side configuration. It is possible that the multicomponent filaments of the first layer are multicomponent filaments with a centric core/sheath configuration. It is also possible that the multicomponent filaments of the third layer or the second nonwoven lamina are multicomponent filaments with side-by-side configuration. If, according to a preferred embodiment, the multicomponent filaments of the first layer are multicomponent filaments with an eccentric core/sheath configuration, it is within the scope of the invention that both the sheath of the filaments and the core/sheath of the filaments in the first layer are multicomponent filaments with an eccentric core/sheath configuration that is circular in cross-section. According to a further preferred embodiment of the invention, the multicomponent filaments of the first layer are multicomponent filaments with an eccentric core/sheath configuration and the core of these filaments is designed in the shape of a segment of a circle as seen in the filament cross-section and has a circular arc-shaped circumferential section and a linear circumferential section with respect to its circumference, so that a D-shape of the core as seen in the filament cross-section results, as it were. If, according to a preferred embodiment of the invention, the multicomponent filaments of the first layer and the third layer or of the second nonwoven lamina are multicomponent filaments with an eccentric core/sheath configuration, it is also preferred that a spacing of the center of gravity of the core from the center of gravity of the sheath in the multicomponent filaments with an eccentric core/sheath configuration of the first layer is smaller than a spacing of the center of gravity of the core from the center of gravity of the sheath of the multicomponent filaments with an eccentric core/sheath configuration of the third layer or the second nonwoven lamina.

It is within the scope of the invention that the base weight of the nonwoven aggregate is 50 to 350 g/m² , preferably 80 to 300 g/m² , preferably 100 to 250 g/m² , based on the aggregate comprising the first layer, second layer and third layer or second nonwoven lamina. In particular, the base weight refers to the aggregate without absorption particles or before the absorption particles are applied and incorporated. Conveniently, the weight of the absorption particles incorporated into the nonwoven aggregate is 200 g/m² to 600 g/m² , preferably 300 g/m² to 500 g/m² , relative to the surface area of the nonwoven aggregate. It has been proven effective when the ratio of the weight of the absorption particles embedded in the nonwoven aggregate in relation to the surface area of the nonwoven aggregate to the base weight of the nonwoven aggregate is 2:1 to 15:1, preferably 3:1 to 8:1 and preferably 3:1 to 5:1. Further preferably, the average titer of the filaments of the aggregate of first layer, second layer and third layer or second nonwoven lamina according to a preferred embodiment of the invention is greater than 2 den, particularly preferably greater than 3 den.

According to a further preferred embodiment of the nonwoven aggregate according to the invention, the filaments of the first layer and the filaments of the second layer and the filaments of the third layer or of the second nonwoven lamina are multicomponent filaments, in particular bicomponent filaments, where the multicomponent filaments have at least one low-melting-point binder and where the average proportion of the low-melting-point binder relative to the components of the multicomponent filaments of the aggregate of first layer, second layer and third layer or second nonwoven lamina is less than 40 wt %, preferably less than 33 wt %. In this context, it is within the scope of the invention that the low-melting-point binder consists or essentially consists of polyethylene. It has been found that the binder content of the multicomponent filaments of the first layer is greater, preferably by at least 5%, than the average binder content of the multicomponent filaments of the first layer, the second layer and the third layer or the second nonwoven lamina.

It is preferred that the mass fraction of the first layer is 5 to 60 wt %, in particular 20 to 50 wt %, preferably 30 to 45 wt %, more preferably the mass fraction of the second layer is 30 to 85 wt %, in particular 35 to 80 wt %, preferably 40 to 75 wt % and further preferably the mass fraction of the third layer or the second nonwoven lamina is 4 to 15 wt %, in particular 5 to 14 wt %, preferably 6 to 13 wt % relative to the aggregate of first layer, second layer and third layer or second nonwoven lamina. In the context of the invention, the stated mass fractions are in particular the mass fractions without absorption particles or before application and incorporation of the absorption particles. Preferably, the mass fraction of the second layer is greater than the mass fraction of the first layer and the third layer and further preferably, the mass fraction of the first layer is greater than the mass fraction of the third layer.

A particularly preferred embodiment of the nonwoven aggregate according to the invention is characterized in that the second layer has at least two, preferably two, partial layers. A first partial layer is preferably adjacent the first layer and a second partial layer is preferably adjacent the third layer or the second nonwoven lamina. Preferably, the second partial layer of the second layer preferably adjacent the third layer or the second nonwoven lamina, has more hydrophilic properties than the first partial layer of the second layer preferably adjacent the first layer. According to a particularly preferred embodiment, the filaments of the first partial layer of the second layer preferably adjacent the first layer, have a lower mean titer than the filaments of the second partial layer of the second layer preferably adjacent the third layer or to the second nonwoven lamina, and/or both partial layers contain multicomponent filaments, the multicomponent filaments of the first partial layer having a higher proportion of binders than the multicomponent filaments of the second partial layer.

It has been proven effective when the average titer of the filaments of the first partial layer is 2 den to 6 den, preferably 2.5 den to 5.5 den, preferably 3 den to 5 den. It is further preferred that the average titer of the filaments of the second partial layer is 2 den to 9 den, preferably 3 den to 8 den, preferably 4 den to 7 den, for example 6 den or about 6 den.

According to a preferred embodiment, the filaments of the first partial layer and/or the second partial layer are multicomponent filaments, in particular bicomponent filaments. Particularly preferred are the filaments of the first partial layer and/or the second partial layer multicomponent filaments with side-by-side configuration. It has been proven effective when the multicomponent filaments of the first partial layer and/or the second partial layer have at least one low-melting-point binder and that this low-melting-point binder consists or essentially consists in particular of polyethylene. Expediently, the binder content of the multicomponent filaments of the first partial layer is 15 to 40 wt %, preferably 20 to 35 wt %, for example 25 wt % or about 25 wt % relative to the components of the multicomponent filaments of the first partial layer. Further preferably, the binder content of the multicomponent filaments of the second partial layer is 5 to 25 wt. %, preferably 10 to 20 wt. %, for example 15 wt. % or about 15 wt. % based on the components of the multicomponent filaments of the second partial layer. Expediently, the further component or the second component of the multicomponent filaments, in particular of the bicomponent filaments, of the first partial layer and/or of the second partial layer consists of polypropylene or essentially of polypropylene.

It is also preferred that the mass fraction of the first partial layer is 15 to 30 wt %, in particular 20 to 30 wt %, and the mass fraction of the second partial layer is 40 to 70 wt %, in particular 50 to 60 wt %, based on the aggregate of first layer, second layer or first and second partial layer of the second layer, and third layer or second nonwoven lamina. The mass fractions here preferably refer to the aggregate without incorporated absorption particles or before the application and incorporation of the absorption particles. It is within the scope of the invention that the average pore size of the pores formed between the filaments of the first partial layer of the second layer is smaller than the average pore size of the pores formed between the filaments of the second partial layer of the second layer. Conveniently, the first partial layer of the first layer and the second partial layer of the third layer or the second nonwoven lamina are adjacent. If the nonwoven aggregate according to an embodiment described above comprises an intermediate layer between the second layer and the third layer or the second nonwoven lamina, it is possible that the intermediate layer has the above-described properties of the second partial layer and that the second layer has the above-described properties of the first partial layer and then has no second partial layer. According to one embodiment, it is also possible that the second layer has no first and second partial layer and that instead the second layer has the above-described properties of the first partial layer of the second layer, where a second nonwoven lamina applied to the second layer comprises two nonwoven layers of continuous filaments, where a first layer of the second nonwoven lamina which is adjacent the second layer has the above-described properties of the second partial layer of the second layer and where the second layer of the second nonwoven lamina has the above-described properties of the third layer or second nonwoven lamina.

It is within the scope of the invention that the nonwoven aggregate has a top layer of continuous filaments above the third layer or the second nonwoven lamina. Preferably, the top layer is a spunbond nonwoven layer and particularly preferably the filaments of the top layer are multicomponent filaments, in particular bicomponent filaments. It has been proven effective when the multicomponent filaments of the top layer are multicomponent filaments with a core/sheath configuration, in particular with an eccentric core/sheath configuration. Preferably, the multicomponent filaments of the top layer have at least one low-melting-point binder component. Further preferably, the low-melting-point binder consists of polyethylene or essentially of polyethylene. The further or second component of the multicomponent filaments or bicomponent filaments of the top layer is expediently made of polypropylene or essentially of polypropylene. It has been proven effective when the binder content of the multicomponent filaments of the top layer is more than 30 wt %, preferably more than 40 wt %, relative to the components of the multicomponent filaments of the top layer. It is further preferred that the average titer of the multicomponent filaments of the top layer is less than 3 den. It is possible that at least one hydrophilizing agent, in particular a hydrophilic lubricant, is applied to the top layer of continuous filaments.

It is preferred that the nonwoven aggregate according to the invention is a consolidated or final-consolidated nonwoven aggregate or nonwoven laminate.

Preferably, the nonwoven aggregate is a thermally bonded or final bonded nonwoven aggregate. It is within the scope of the invention that the consolidation or final consolidation, in particular the thermal consolidation or final consolidation, of the nonwoven aggregate is carried out with the aid of a consolidator, in particular with the aid of a calender. According to one embodiment, the consolidator can have a flow-through oven and/or a hot calender. It is advisable to feed the aggregate pre-bonded in the oven directly to the hot calender without significant cooling in order to utilize the product waste heat for effective hot calendering.

According to a particularly preferred embodiment of the nonwoven aggregate according to the invention, the absorption particles are embedded in the second layer in the form of lines or essentially in the form of lines, the lines preferably being oriented or essentially oriented in the machine direction (MD direction). It is preferred that the lines of absorption particles have the same spacing or essentially the same spacing in the direction transverse to the machine direction (CD direction).

According to a particularly preferred embodiment of the nonwoven aggregate according to the invention, the liquid strike-through time from the hydrophilic outer face or upper face of the nonwoven aggregate during the first exposure to a test liquid is less than 30 s, preferably less than 15 s, particularly preferably less than 10 s and very particularly preferably less than 5 s. Hydrophilic outer face means the outer face or upper face of the nonwoven aggregate that has more hydrophilic properties than the opposite outer face or lower side of the nonwoven aggregate. According to one embodiment of the invention, the more hydrophilic nonwoven aggregate outer face is formed by the third layer or the second nonwoven lamina. The liquid strike-through time is determined in the context of the invention in particular in accordance with the laboratory test EDANA Harmonized Nonwovens Standard Procedures “Repeated Liquid Strike-Through Time (Simulated Urine) ” NWSP 070.7.R2(20). It is preferred that the liquid strike-through time from the hydrophilic or more hydrophilic outer face or upper face of the nonwoven aggregate during repeated exposure (second to fourth exposure to a test liquid) is less than 30 s, preferably less than 15 s, particularly preferably less than 10 s and very particularly preferably less than 5 s.

According to a preferred embodiment, the nonwoven aggregate according to the invention is characterized in that the rewet mass is less than 20 g, preferably less than 15 g, particularly preferably less than 10 g and most preferably less than 5 g. In the context of the invention, the rewet mass is determined in accordance with the laboratory test EDANA Harmonized Nonwovens Standard Procedures “Wetback After Repeated Strike-Through Time (Simulated Urine)” NWSP 070.8.R0 (15) or “Wetback After Repeated Strike-Through Time (Simulated Urine)” NWSP 070.8.R1 (19).

To attain these objects, the invention further teaches a method of making a nonwoven aggregate, in particular a nonwoven aggregate as described above, where at least a first layer is deposited on a receiving surface, in particular on a mesh deposition belt, and where at least a second layer in the form of a nonwoven layer of continuous filaments is produced by spinning out the continuous filaments from a spinning beam and is deposited on the first layer, where at least a third layer in the form of a nonwoven layer of continuous filaments or at least a second nonwoven lamina of continuous filaments is deposited on the second layer, wherein
the first layer is produced such that it forms a barrier against penetration by particles or absorption particles with a size greater than 150 μm, preferably greater than 100 μm, preferably greater than 50 μm, very preferably greater than 25 μm, particularly preferably greater than 10 μm and very particularly preferably greater than 0.5 μm,
absorption particles are applied to the aggregate before and/or after the deposition of the third layer or the second nonwoven lamina, the absorption particles being embedded between the filaments of the second layer, and
at least one hydrophilizing agent, in particular a hydrophilic lubricant, is applied to the aggregate before and/or after the deposition of the third layer or the second nonwoven lamina, in particular after the application and incorporation of the absorption particles onto or into the aggregate.

A particularly preferred embodiment of the process according to the invention is characterized in that the first layer is also produced as a nonwoven layer of continuous filaments and the filaments for the first layer and the filaments for the second layer are spun with the same spinning beam.

According to a Particularly Preferred Embodiment of the method according to the invention, the filaments for the first layer and the filaments for the second layer and the filaments for the third layer are spun with the same spinning beam. Then, expediently, at least one spinning-beam property or at least one filament property from the group: “per-hole throughput, cross-sectional geometry of the multicomponent filaments, binder proportion of the multicomponent filaments” is varied on this spinning beam in the machine direction (MD direction) in order to produce the three mutually different layers or to deposit them on top of one another. Conveniently, viewed in the MD direction, the inlet side of the spinning beam is provided for making the first layer, the middle area for making the second layer and the outlet side for making the third layer. The three areas of the spinning beam intended for making the first layer, the second layer and the third layer then produce the three layers with the properties of the filaments described above. In this way, the properties of the layers can be set very precisely and accurately, resulting in a nonwoven aggregate in which properties such as the average pore size can be set very precisely and in fine increments. The selection and combination of spinning-beam properties or filament properties within a spinning beam is, as it were, a toolbox for making nonwoven layers from continuous filaments with precisely and finely adjustable and tunable properties. It is alternatively possible within the framework of the method according to the invention for the third layer to be produced by partial consolidation, in particular by thermal partial consolidation, of the outer face of the second layer directed toward the outside of the nonwoven aggregate.

According to an alternative embodiment of the method according to the invention, a second nonwoven lamina of continuous filaments is deposited on the second layer and this second nonwoven lamina of continuous filaments is spun with a separate spinning beam. It is possible for the second nonwoven lamina to be unwound from an unwinder or a roll and placed on the second layer. It is also possible that, as part of the method according to the invention, a top layer of continuous filaments is spun using a further separate spinning beam and is deposited on the third layer or on the second nonwoven lamina or on the second layer. It is possible for the top layer to be unwound from an unwinder or a roll and placed on the third layer or the second nonwoven lamina or on the second layer. When the second nonwoven lamina is deposited, the first layer and the second layer are preferably compacted by the process air of a second spinning beam. In the process according to the invention, this preferably makes it possible to reduce the average pore size of the first and second layers during the deposition of the second nonwoven lamina.

According to the invention, absorption particles are applied to the aggregate before and/or after the deposition of the third layer or the second nonwoven lamina. Expediently, the absorption particles are applied or deposited on the second layer and/or on the third layer or the second nonwoven lamina. According to one embodiment of the method according to the invention, the absorption particles are applied to the second layer immediately after the second layer has been deposited and then the third layer or the second nonwoven lamina is deposited on the second layer. According to a further preferred embodiment of the method according to the invention, the absorption particles are applied to the third layer or the second nonwoven lamina immediately after the third layer or the second nonwoven lamina has been deposited.

According to a preferred embodiment of the method according to the invention, the third layer or the second nonwoven lamina is produced such that absorption particles applied to the third layer or the second nonwoven lamina penetrate the third layer or the second nonwoven lamina at least before the third layer or the second nonwoven lamina is consolidated and/or compacted and are at least for the most part embedded between the filaments of the second layer. It is within the scope of the invention that after the absorption particles have been applied, in particular after the absorption particles have been applied to the second layer and/or to the third layer or the second nonwoven lamina, the absorption particles are introduced into the nonwoven aggregate. Injecting the absorption particles into the nonwoven aggregate or into the second layer is expediently effected by mechanical energy and/or by an electric field and/or by acoustic pressure, for example by sound. Preferably, the absorption particles are introduced into the nonwoven aggregate or into the second layer by acoustic pressure in the range from 100 Hz to 25 kHz, preferably in the range from 10 kHz to 18 kHz, particularly preferably in the range from 14 kHz to 16 kHz. The amplitude of the acoustic pressure is also preferably between 5 μm and 5000 μm, preferably between 50 μm and 150 μm and particularly preferably between 80 μm and 110 μm.

According to the invention, at least one hydrophilizing agent, in particular a hydrophilic lubricant, is applied to the aggregate before and/or after the deposition of the third layer or the second nonwoven lamina, in particular after the application and incorporation of the absorption particles onto or into the aggregate. The at least one hydrophilizing agent or the hydrophilic lubricant is preferably applied to or into the aggregate after the absorption particles have been applied and incorporated.

In principle, however, the hydrophilizing agent can also be applied before the absorption particles are applied to or introduced into the aggregate.

It is possible that the at least one hydrophilizing agent is applied or applied to the second layer. Additionally or alternatively, it is possible that the hydrophilizing agent is applied or applied to the third layer or the second nonwoven lamina. Preferably, a drying step takes place after the application of the at least one hydrophilizing agent or the hydrophilic lubricant and in particular immediately after the application of the hydrophilizing agent or the hydrophilic lubricant. In the drying step, the hydrophilizing agent or the hydrophilic lubricant is preferably dried. In the context of the invention, the at least one hydrophilizing agent or the at least one hydrophilic lubricant is a liquid hydrophilizing agent or a liquid hydrophilic lubricant. It is possible that the drying of the hydrophilizing agent or the hydrophilic lubricant is carried out by a device for consolidating the nonwoven aggregate. According to a further embodiment, the drying of the applied hydrophilizing agent or the applied hydrophilic lubricant can be carried out in an oven.

It is preferred that the application of the at least one hydrophilizing agent or the hydrophilic lubricant is carried out by spray application. It is also possible that the at least one hydrophilizing agent or the hydrophilic lubricant is applied by a roller application (kiss roll). In principle, it is possible for the application of the at least one hydrophilizing agent or the hydrophilic lubricant to be carried out by an immersion bath. Preferably, the application of the at least one hydrophilizing agent or the hydrophilic avivage is carried out by an immersion bath.

Preferably, the application of the at least one hydrophilizing agent or the hydrophilic avivage as part of the process according to the invention is by spray application. This embodiment has proven to be particularly effective.

It is within the scope of the method according to the invention that, after the injecting the absorption particles into the nonwoven aggregate or into the second layer, a consolidation of the surface or of the third layer or of the second nonwoven lamina and/or a consolidation of the entire aggregate comprising the first layer, second layer and third layer or second nonwoven lamina takes place. The consolidating of the third layer or the second nonwoven lamina after the injecting the absorption particles into the nonwoven aggregate or into the second layer preferably leads to a sealing of this layer, so that the absorption particles can first be introduced into the nonwoven aggregate through this layer or lamina and this sealed layer or lamina then acts as a barrier for the absorption particles.

It is also possible within the framework of the method according to the invention that, after the production and deposition of the first layer and the second layer and the third layer or the second nonwoven lamina, a preconsolidation or compaction of the deposited aggregate takes place and that the application and injection of the absorption particles and/or the application of the hydrophilic lubricant is then carried out. According to a further preferred embodiment, pre-consolidation or compaction of the aggregate comprising the first layer and the second layer and the third layer or the second nonwoven lamina takes place after the application of the hydrophilizing agent or the hydrophilic lubricant. This is particularly advantageous since the process heat of the preconsolidation or compaction can dry the residual moisture of the hydrophilic lubricant without additional effort. It is also within the scope of the invention that no pre-consolidation of the nonwoven aggregate and that the entire nonwoven aggregate is only bonded or finally bonded after the application and injection of the absorption particles into the nonwoven aggregate and after the application of the hydrophilizing agent or the hydrophilic lubricant.

As part of the method according to the invention, it is possible for the aggregate comprising the first layer and the second layer and the third layer or the second nonwoven lamina to be consolidated with as little compaction as possible before the absorption particles are applied, such that the nonwoven aggregate is transportable. After this consolidating of the nonwoven aggregate comprising the first layer and the second layer and the third layer or the second nonwoven lamina, the nonwoven aggregate can initially be laid down or wound up so that the application and injection of the absorption particles into the nonwoven aggregate can take place “offline” in a separate step. In such an embodiment, the application of the at least one hydrophilizing agent or the hydrophilic lubricant can take place before or after the application and injection of the absorption particles.

It is within the scope of the method according to the invention that the nonwoven aggregate is cut to size for later use. Sufficient immobilization of the absorption particles is intended to ensure that the absorption particles can be held or fixed in the nonwoven aggregate despite the cutting. This is conveniently achieved by sealing and/or welding the cut surface. The sealing and/or welding can be carried out by thermal and/or thermomechanical and/or mechanical treatment, in particular by a laser, a fluid, hot stamping or ultrasound. Preferably, the sealing or welding takes place in a process step combined with the cutting. In the case of a laser cut, the process parameters can be selected so that the edge areas melt and fuse and welded cut surfaces are formed on both sides of the cut on the nonwoven aggregate. This can also be done with an appropriately designed ultrasonic sonotrode, for example in the form of a roller.

To attain these objects, the invention further teaches an apparatus for making a nonwoven aggregate, in particular for making a nonwoven aggregate as described above and/or produced using a method described above, where a spinning beam is provided for spinning out continuous filaments and a receiving surface, in particular a mesh deposition mesh deposition belt, is provided for deposition of the filaments to form a nonwoven web, where filaments for at least a first layer or first nonwoven layer can be produced with the spinning beam and can be deposited on the receiving surface, where filaments for at least a second layer or second nonwoven layer can be produced with the same spinning beam, which filaments can be deposited on the first layer or the first nonwoven layer, the spinning beam or the spinneret openings of the spinning beam being set up such that
the filaments of the first layer have a lower mean titer than the filaments of the second layer
and/or
the filaments of the first and second layers are crimped multicomponent filaments and the filaments of the second layer have a higher degree of crimp than the filaments of the first layer
and/or
the filaments of the first and second layers are multicomponent filaments with a low-melting-point binder component and the binder component content of the filaments of the first layer is higher than the binder component content of the filaments of the second layer,
filaments for at least a third layer or third nonwoven layer can be produced with the spinning beam and can be deposited on the second layer, or wherein the apparatus has a second spinning beam for spinning out filaments for a second nonwoven lamina or an unwinder for unwinding a second nonwoven lamina,
an application and/or injection device for applying and/or injecting absorption particles onto or into the aggregate is downstream of the spinning beam in the machine direction, and
at least one applicator for applying at least one hydrophilizing agent, in particular a hydrophilic lubricant, to the aggregate is downstream of the spinning beam in the machine direction.

It is possible for the apparatus according to the invention to have an unwinder for applying a top layer to the third layer or the second nonwoven lamina. The unwinder for applying the top layer is expediently downstream to the first spinning beam or the second spinning beam. If the apparatus has an unwinder for a top layer, it is preferred that the apparatus has an oven for thermally consolidating the top layer to the third layer or the second nonwoven lamina. The apparatus according to the invention has at least one applicator for applying at least one hydrophilizing agent, in particular a hydrophilic lubricant, to the aggregate. According to a preferred embodiment, an applicator is a spray system, for example as a spray nozzle. The applicator or the spray system is then provided in particular for spray application of the at least one hydrophilizing agent or the hydrophilic lubricant. It is also possible that an applicator is an application roller. In this case, the applicator or the application roller is intended in particular for the roller application of the at least one hydrophilizing agent or the hydrophilic lubricant (kiss-roll application).

The invention is based on the discovery that the nonwoven aggregate according to the invention enables optimum transport of a fluid medium from an outer or upper face of the nonwoven aggregate to absorption particles stored within the nonwoven aggregate. Furthermore, the nonwoven aggregate according to the invention effectively and reliably prevents further passage of the fluid medium through the nonwoven aggregate and, in particular, prevents the fluid medium from escaping from the other nonwoven aggregate outer face or lower face. The nonwoven aggregate according to the invention thus very reliably enables the passage of a fluid medium to the embedded absorption particles and prevents the further passage of the fluid medium and its escape from the nonwoven aggregate. In addition, the absorption particles can be very securely fixed in the nonwoven aggregate according to the invention, so that movement or displacement of the absorption particles in the nonwoven aggregate according to the invention can be largely avoided. According to a preferred embodiment, a particle-size gradient of the embedded absorption particles is also realized across the thickness of the nonwoven aggregate, at least in certain areas or sections. The nonwoven aggregate according to the invention thus ensures both a functionally reliable fixation of the absorption particles and also enables optimum fluid transport into the nonwoven aggregate. Compared to the known measures, these properties can be realized very reliably and with little effort. Furthermore, with the nonwoven aggregate according to the invention, a very precise gradation of the properties of the layers, for example with regard to their hydrophilicity and/or with regard to their average pore size, is possible.

The invention is described in the following in more detail with reference to a drawing showing only one embodiment.

Therein:

FIG. 1 is a sectional view of a nonwoven aggregate according to the invention in a first embodiment,

FIG. 2 is a sectional view of a nonwoven aggregate according to the invention in a second embodiment,

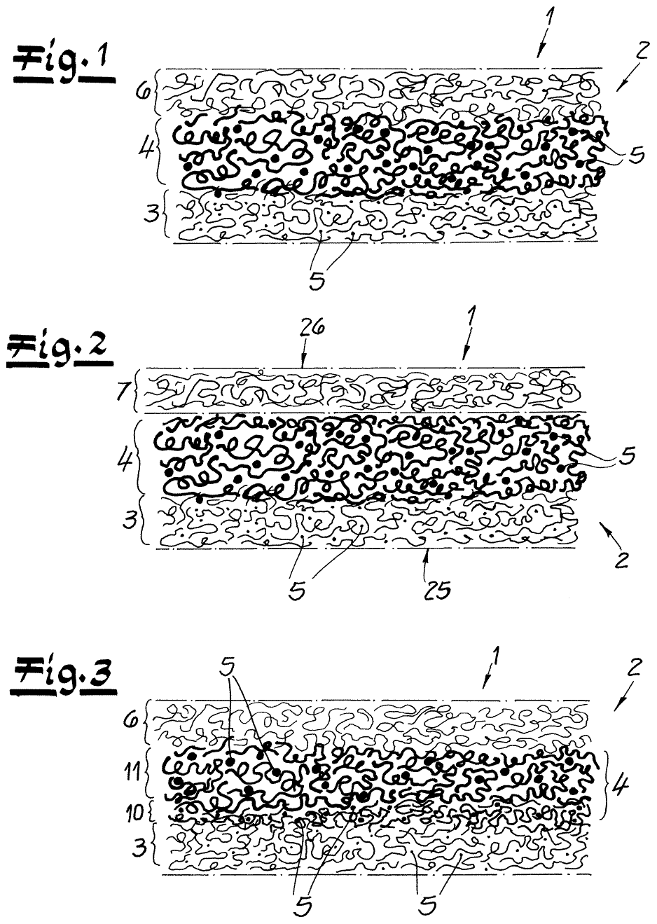

FIG. 3 is a sectional view of a nonwoven aggregate according to the invention in a third embodiment,

FIG. 4: a vertical section through an apparatus according to the invention for making a nonwoven aggregate according to the invention,

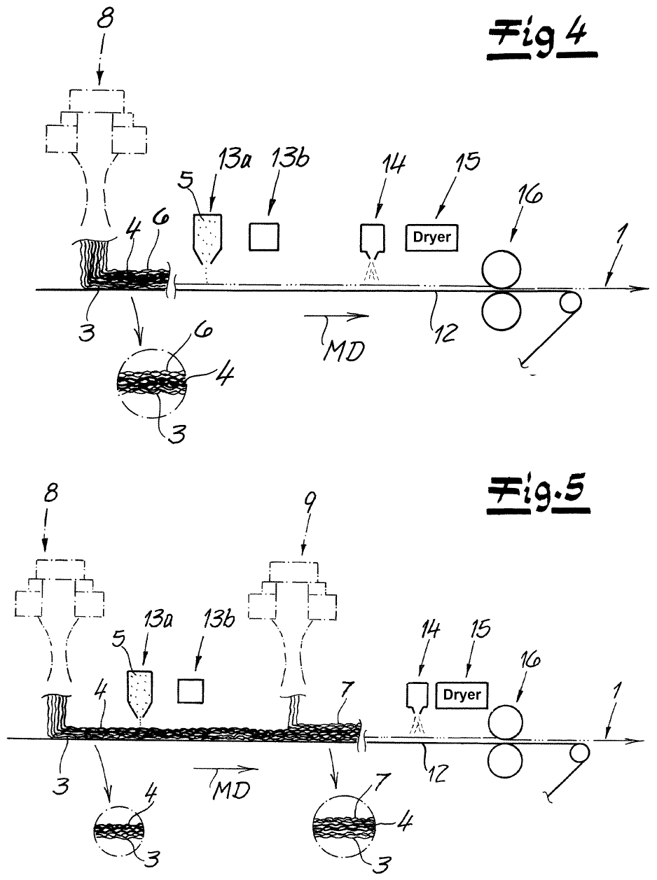

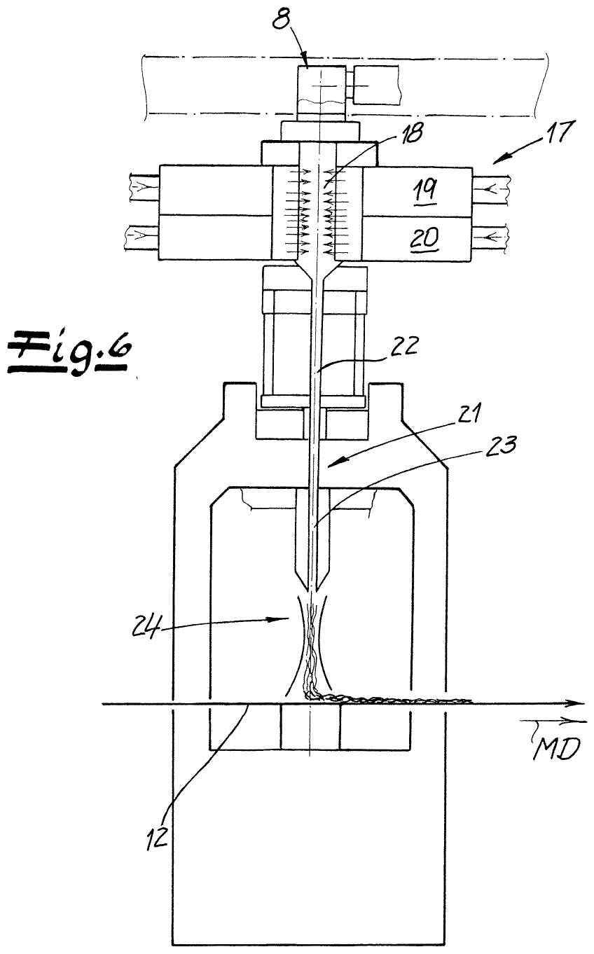

FIG. 5 is a vertical section through an apparatus according to the invention for making a nonwoven aggregate according to the invention in a second embodiment,

FIG. 6 is a vertical section through a part of the apparatus according to the invention for making a layer or spunbond layer.

FIG. 1 shows a nonwoven aggregate 1 according to the invention with a nonwoven lamina 2 with at least a first layer 3 and at least a second layer 4. Preferably and here, the first layer 3 and the second layer 4 are a spunbond nonwoven layer of continuous filaments. The nonwoven aggregate 1 according to FIG. 1 also preferably and here has a third layer 6 applied directly to the second layer 4 in the form of a spunbond nonwoven layer of continuous filaments. The second layer 4 has functional particles 5 embedded in the pores between the filaments of the second layer 4. The average pore size of the pores formed in the first layer 3 is smaller than the average pore size of the pores between the filaments of the second layer 4. Conveniently and here, functional particles 5 are also embedded in the pores between the filaments of the first layer 3 whose average size is preferably and here smaller than the average size of the functional particles 5 embedded in the pores of the second layer 4. Preferably and here, the functional particles 5 are embedded in the pores of the second layer 4 without adhesives or binders. Moreover and according to a preferred embodiment, this also applies to the functional particles 5 that are embedded in the pores of the first layer 3. The functional particles 5 may be absorption particles, in particular for absorbing fluid media.

The nonwoven lamina 2 or the first layer 3 and the second layer 4 and the third layer 6 are particularly preferably and here according to FIG. 1 spun from only one spinning beam or from only one spinneret. Preferably and here according to FIG. 1, the filaments of the first layer 3 and the filaments of the third layer 6 have a lower mean titer than the filaments of the second layer 4. Further preferably and here, the filaments of the third layer 6 have a higher mean titer than the filaments of the first layer 3. Here according to FIG. 1, the titer of the filaments of the first layer 3 may be about 2.0 den, the mean titer of the filaments of the second layer 4 may be about 6.0 den and the mean titer of the filaments of the third layer 6 may be about 2.5 den. The filaments of the first layer 3 and the second layer 4 and the third layer 6 are preferably and here crimped bicomponent filaments, the bicomponent filaments having a low-melting-point binder component and a high-melting-point second component. Here according to the figures, the low-melting-point binder may consist of or essentially consist of polyethylene and the second component or the high-melting-point component may consist of or essentially consist of polypropylene.

Preferably and here, the degree of crimp of the filaments of the first layer 3 and the filaments of the third layer 6 is lower than the degree of crimp of the filaments of the second layer 4.

According to a preferred embodiment and here according to the figures, the mass fraction of the first layer 3 is 5 to 60 wt % and the mass fraction of the second layer 4 is 30 to 85 wt % and the mass fraction of the third layer 6 is 5 to 15 wt % relative to the aggregate of first layer 3, second layer 4 and third layer 6. The mass fractions preferably and here relate to the aggregate without functional particles or before the application and incorporation of the functional particles. Here according to the figures, the mass fraction of the first layer 3 may be 20 wt %, the mass fraction of the second layer 4 may be 70 wt % and the mass fraction of the third layer 6 may be 10 wt %, based on the aggregate of first layer 3, second layer 4 and third layer 6.

Preferably, the third layer 6 has hydrophilic properties and, according to a preferred embodiment and here, has more hydrophilic properties than the first layer 3. For this purpose, preferably and here according to FIG. 1, a hydrophilizing agent, in particular in the form of a hydrophilic avivage, is applied or applied to the third layer 6.

FIG. 2 shows a further embodiment of the nonwoven aggregate according to the invention. In the embodiment shown in FIG. 2, a second nonwoven lamina 7 of continuous filaments is present instead of a third nonwoven layer of continuous filaments. With regard to the layers or laminae, essentially the same description applies as for FIG. 1. The second nonwoven lamina 7 is spun from a further or second spinning beam 9 or a further or second spinneret and deposited on the second layer 4. Preferably and here, the second nonwoven lamina 7 is a spunbond nonwoven layer. Conveniently and here according to FIG. 2, the second nonwoven lamina 7 has more hydrophilic properties than the first layer 3. Further preferably and here, the second nonwoven lamina 7 has more hydrophilic properties than the second layer 4. Conveniently and here according to FIG. 2, the hydrophilic properties of the nonwoven aggregate 1 increase across the thickness of the nonwoven aggregate from the first layer 3 to the second nonwoven lamina 7 and thus in particular from a first nonwoven aggregate outer face 25 or lower face to a second nonwoven aggregate outer face 26 or upper face opposite the first nonwoven aggregate outer face 25.

FIG. 3 shows a further embodiment of a nonwoven aggregate 1 according to the invention. In the embodiment according to FIG. 3, the nonwoven aggregate 1 has, analogous to the embodiment according to FIG. 1, a first layer 3 in the form of a nonwoven layer of continuous filaments, a second layer 4 in the form of a nonwoven layer of continuous filaments and a third layer 6 in the form of a nonwoven layer of continuous filaments, where the first layer 3 and the second layer 4 and the third layer 6 are spun from the same spinning beam 8 or from the same spinneret. The first layer 3 and the second layer 4 and the third layer 6 are preferably and here spunbond nonwoven layers. In the embodiment according to FIG. 3 and preferably, the second layer 4 consists of two partial layers 10, 11, where the first partial layer 10 is adjacent the first layer 3 and where the second partial layer 11 is adjacent the third layer 6. In this way, the properties of the second layer 4 can be particularly finely adjusted or graded. In the embodiment according to FIG. 3 with the two partial layers 10, 11 of the second layer 4, the filaments of the first partial layer 10 of the second layer 4 preferably have a lower mean titer than the filaments of the second partial layer 11 of the second layer 4.

Furthermore, preferably and here, the mean pore size of pores formed between the filaments of the first partial layer 10 is smaller than the mean pore size of pores formed between the filaments of the second partial layer 11. However, the mean pore size of pores formed between the first partial layer 10 is preferably and here larger than the mean pore size of pores formed between the filaments of the first layer 3 and the third layer 6. FIG. 3 also shows that there is a particle-size gradient within the second layer 4 with respect to the average size of the embedded absorption particles 5. Conveniently and here, the average particle size of the embedded particles decreases from the second partial layer 11 toward the first partial layer 10 and further toward the first layer 3. The proportion by mass of the first partial layer 10 is preferably 15 to 30 wt % relative to the aggregate of first layer 3, second layer 4 and third layer 6 and may be about 20 wt % here. The mass fraction of the second partial layer 11 is preferably 40 to 70 wt % relative to the aggregate of first layer 3, second layer 4 and third layer 6 and may be about 50 wt % here.

Preferably, the third layer 6 has hydrophilic properties and, particularly preferably and here according to FIG. 3, has more hydrophilic properties than the first layer 3. For this purpose, preferably and here according to FIG. 3, at least one hydrophilizing agent, in particular in the form of a hydrophilic lubricant, is applied to the third layer 6.

FIG. 4 shows an apparatus according to the invention for making a nonwoven aggregate 1 according to the invention in a first embodiment. The apparatus has a spinning beam 8 for spinning continuous filaments, as well as a mesh deposition belt forming a receiving surface 12 for deposition of the filaments to form a nonwoven web. Preferably and here, filaments for a first layer 3 in the form of a nonwoven layer of continuous filaments can be produced with the spinning beam 8 and deposited on the receiving surface 12. Preferably and here, the same spinning beam 8 can be used to produce filaments for a second layer 4 in the form of a nonwoven layer of continuous filaments, which filaments can be deposited on the first layer 3. Preferably and here, the same spinning beam 8 can be used to produce filaments for a third layer 6 in the form of a nonwoven layer of continuous filaments, which can be deposited on the second layer. The spinning beam 8 or the spinneret openings of the spinning beam 8 are preferably and here set up such that the filaments of the first layer 3 have a lower mean titer than the filaments of the second layer 4 and that the filaments of the third layer 6 have a higher mean titer than the filaments of the first layer 3, but a lower mean titer than the filaments of the second layer 4. Further preferably and here, the spinning beam 8 or the spinneret openings of the spinning beam 8 are dimensioned such that the filaments of the first layer 3 and of the second layer 4 and of the third layer 6 are crimped multicomponent filaments and the filaments of the second layer 4 have a higher degree of crimp than the filaments of the first layer 3 and the filaments of the third layer 6.

Preferably and here, the apparatus has a device 13a, 13b for applying and injecting absorption particles 5 onto and into the aggregate downstream of the spinning beam 8 in the machine direction (MD direction). Preferably and here, the applying and injecting unit 13a, 13b has an applicator 13a and an injector 13b. The applying and injecting unit 13a, 13b can preferably apply the functional particles to the aggregate and introduce them into the aggregate. The applicator 13a is preferably and here a scattering funnel. The injector 13b may be a device for generating acoustic pressure, for example by sound. Preferably and here, the apparatus according to the invention also has an applicator 14, in particular a spray nozzle, for applying a hydrophilic lubricant to the nonwoven aggregate. The applicator 14 is expediently downstream of the spinning beam 8 in the machine direction and downstream of the applying and injecting unit 13a, 13b for applying and injecting the absorption particles 5. Further preferably and here, a dryer 15 for drying the applied or sprayed-on hydrophilic lubricant is provided downstream of the applicator 14 in the machine direction. The apparatus according to the invention expediently and here has a consolidator 16 for consolidating the nonwoven aggregate. This consolidator 16 can have a flow-through oven and/or a hot calender. It is advisable to feed the aggregate prebonded in the oven directly to the hot calender without significant cooling in order to utilize the product waste heat for effective hot calendering.

FIG. 5 shows a second embodiment of the apparatus according to the invention. For this second embodiment, essentially the same description applies as for FIG. 4. However, the apparatus according to the second embodiment has for making continuous filaments a second spinning beam 9 downstream of the first spinning beam 8 in the machine direction and upstream of the applicator 14 for applying a hydrophilic lubricant to the nonwoven aggregate in the machine direction. With the apparatus shown in FIG. 5, a first layer 3 in the form of a nonwoven layer of continuous filaments and a second layer 4 in the form of a nonwoven layer of continuous filaments can be produced with the first spinning beam 8. A second nonwoven lamina 7 of continuous filaments can be deposited on the aggregate of the first layer 3 and the second layer 4 with the apparatus shown in FIG. 5 spun with the second spinning beam 9. The subsequent application of the hydrophilic lubricant by the applicator 14 and drying of the hydrophilic lubricant by the dryer 15 is carried out in the same way as in the embodiment shown in FIG. 4. This also applies to the consolidating of the nonwoven aggregate with the consolidator 16. In the apparatus according to FIG. 5 and according to the preferred embodiment, a unit 13a, 13b for applying and injecting absorption particles 5 onto and into the aggregate is provided downstream of the spinning beam 8 in the machine direction and upstream of the spinning beam 9 in the machine direction. The applying and injecting unit 13a, 13b is thus preferably and here, between the spinning beam the two spinning beams 8, 9. Preferably and here, the application and the injection device 13a, 13b has an applicator 13a formed as a scattering funnel and an injector 13b preferably and here a device for generating acoustic pressure.

FIG. 6 shows the basic structure of a part of the apparatus according to the invention for making a layer or spunbond layer for the nonwoven aggregate according to the invention using the spunbond method, comprising a spinneret or the spinning beam 8 for spinning the continuous filaments for a nonwoven layer of continuous filaments. The continuous filaments spun by the spinneret or the spinning beam 8 are introduced into a cooler 17 with a cooling chamber 18. Preferably and here, air supply cabins 19, 20 are one above the other on two opposite sides of the cooling chamber 18. Air of different temperatures is preferably introduced into the cooling chamber 18 from the air supply cabins 19, 20 one above the other.

In a recommended embodiment, a stretcher 21 for stretching the continuous filaments is downstream of the cooler 3 toward filament flow. Expediently and here, the stretcher 21 has an intermediate passage 22 that connects the cooler 17 to a stretching shaft 23 of the stretcher 21. Preferably and here, the unit comprising the cooler 17 and the intermediate passage 22 and the stretching shaft 23 are a closed unit and apart from the supply of cooling air in the cooler 17, no further air is supplied to this unit from outside.

Conveniently and here, a diffuser 24 follows the stretcher 21 in the filament flow direction, through which the continuous filaments are guided. After passing through the diffuser 24, the continuous filaments are preferably and here deposited on a receiving surface 12 formed as a mesh deposition mesh deposition belt. The mesh deposition mesh deposition belt is preferably and here an endlessly circulating mesh deposition mesh deposition belt. It is within the scope of the invention that the mesh deposition belt is permeable to air, so that process air can be extracted from below through the mesh deposition belt.

1. A nonwoven aggregate comprising at least one nonwoven lamina comprising in turn:
at least one first layer;
at least one second layer that is a nonwoven layer of continuous filaments and has absorption particles embedded in pores between the filaments;
at least one third layer in the form of a nonwoven layer of continuous filaments applied to the second layer or at least one second nonwoven lamina of continuous filaments applied to the second layer, the third layer or the second nonwoven lamina having hydrophilic properties, the first layer forming a barrier against penetration by particles or absorption particles with a size greater than 150 μm.

2. The nonwoven aggregate according to claim 1, wherein the mean pore size of pores formed in the first layer is smaller than the mean pore size of the pores between the filaments of the second layer and/or the third layer or the second nonwoven lamina has a mean pore size of the pores between the filaments that is smaller than the mean pore size of the pores between the filaments of the second layer.

3. The nonwoven aggregate according to claim 1, wherein the third layer or the second nonwoven lamina forms a barrier against penetration by particles or absorption particles with a size greater than 150 μm.

4. The nonwoven aggregate according to claim 1, wherein the absorption particles are embedded in the pores of the second layer without adhesives or binders.

5. The nonwoven aggregate according claim 1, wherein the third layer or the second nonwoven lamina has more hydrophilic properties than the second layer and the hydrophilic properties of the nonwoven aggregate increase across the thickness of the nonwoven aggregate from a first nonwoven aggregate outer face to a second nonwoven aggregate outer face opposite the first nonwoven aggregate outer face.

6. The nonwoven aggregate according to claim 1, further comprising:
at least one hydrophilizing agent applied to the third layer or the second nonwoven lamina and/or to the second layer.

7. The nonwoven aggregate according to claim 1, wherein the first layer is a nonwoven layer of continuous filaments and the first layer and/or the second layer and/or the third layer is a spunbond nonwoven layer and/or the second nonwoven lamina is a spunbond nonwoven layer.

8. The nonwoven aggregate according to claim 1, wherein the first nonwoven layer or the first layer and the second layer are spun from only one spinning beam or from only one spinneret.

9. The nonwoven aggregate according to claim 8, wherein the third layer is spun from the same spinning beam or from the same spinneret as the first and the second layer or wherein the second nonwoven lamina is spun from a further or second spinning beam or from a further or second spinneret.

10. The nonwoven aggregate according to claim 7, wherein the filaments of the first layer and/or the filaments of the third layer or the second nonwoven lamina have a lower average titer than the filaments of the second layer and the filaments of the third layer or the second nonwoven lamina have a higher average titer than the filaments of the first layer.

11. The nonwoven aggregate according to claim 1, wherein the filaments of the first layer and/or the filaments of the second layer and/or the filaments of the third layer or of the second nonwoven lamina are multicomponent filaments and the multicomponent filaments contain at least one low-melting-point binder.

12. The nonwoven aggregate according to claim 1, wherein the degree of crimp of the filaments of the first layer and/or the filaments of the third layer or second layer is lower than the degree of crimp of the filaments of the second layer.

13. The nonwoven aggregate according to claim 11, wherein the proportion of the low-melting-point binder in the multicomponent filaments of the first layer and/or the third layer or second nonwoven lamina is higher than the proportion of the low-melting-point binder in the multicomponent filaments of the second layer.

14. The nonwoven aggregate according claim 11, wherein the binder proportion of the multicomponent filaments of the second layer is such that when the absorption particles swell and at least partial breaking of the bonds between the filaments of the second layer is possible, the binder proportion of the multicomponent filaments being 5 to 55 wt % based on the components of the multicomponent filaments.

15. The nonwoven aggregate according to claim 11, wherein the multicomponent filaments of the first layer and/or of the third layer or second nonwoven lamina are multicomponent filaments with a core/sheath configuration.

16. The nonwoven aggregate according to claim 1, wherein the mass fraction of the first layer is 5 to 60 wt %, the mass fraction of the second layer is 30 to 85 wt %, and the mass fraction of the third layer or the second nonwoven lamina is 4 to 15 wt % based on the aggregate of the first layer and the second nonwoven lamina.

17. The nonwoven aggregate according to claim 1, wherein the second layer has first and second partial layers, the second partial layer of the second layer or the second nonwoven lamina is more hydrophilic than the first partial layer of the second layer, and/or the filaments of the first partial layer of the second layer have a lower average titer than the filaments of the second partial layer of the second layer and/or both partial layers contain multicomponent filaments, and the multicomponent filaments of the first partial layer have a higher binder content than the multicomponent filaments of the second partial layer.

18. The nonwoven aggregate according to claim 1, wherein the absorption particles are embedded in the second layer in a linear or substantially linear manner.

19. A method of making a nonwoven aggregate, comprising the steps of:
depositing at least a first layer on a mesh deposition belt;
spinning continuous filaments with a spinning beam and depositing the filaments as a second nonwoven layer on the first layer;
depositing at least a third nonwoven layer of continuous filaments or at least a second nonwoven lamina of continuous filaments on the second layer, whereby the first layer forms a barrier against penetration by particles or absorption particles with a size greater than 150 μm;
applying absorption particles to the aggregate before and/or after deposition of the third layer or the second nonwoven lamina such that the absorption particles are embedded between the filaments of the second layer; and, before and/or after the depositing the third layer (6) or the second nonwoven lamina
applying a hydrophilic hydrophilizing agent to the aggregate.

20. The method according to claim 19, wherein the first layer is also produced as a nonwoven layer of continuous filaments from the same spinning beam as for the filaments for the second layer.

21. The method according to one of claims 19, wherein the third layer or the second nonwoven lamina is produced such that absorption particles applied to the third layer or the second nonwoven lamina penetrate the third layer or the second nonwoven lamina at least before consolidation and/or compaction of the third layer or second nonwoven lamina and are embedded at least for the most part between the filaments of the second layer.

22. An apparatus for making a nonwoven aggregate the apparatus comprising:
a spinning beam for spinning endless filaments and depositing the spun endless filaments on a mesh deposition belt to form a nonwoven web, the spinning beam making filaments for at least one first nonwoven layer and depositing same on the mesh belt, the same spinning beam making filaments for at least one second layer or second nonwoven layer that are deposited on the first layer or first nonwoven layer, and the spinning beam or the spinneret openings of the spinning beam being set up such that the filaments of the first layer have a lower mean titer than the filaments of the second layer
and/or
the filaments of the first and second layers are crimped multicomponent filaments and the filaments of the second layer have a higher degree of crimp than the filaments of the first layer
and/or
the filaments of the first and second layers are multicomponent filaments with a low-melting-point binder component and the binder component content of the filaments of the first layer is higher than the binder component content of the filaments of the second layer,
filaments for at least a third layer or third nonwoven layer can be produced with the spinning beam and can be deposited on the second layer or the apparatus has a second spinning beam for spinning out filaments for a second nonwoven lamina or an unwinder for unwinding a second nonwoven lamina,
at least one device for applying and/or injecting absorption particles onto or into the aggregate is downstream of the spinning beam in the machine direction, and
at least one applicator for applying at least one hydrophilizing agent, in particular a hydrophilic lubricant, to the aggregate is provided downstream of the spinning beam in the machine direction.
